(19) United States
(12) Patent Application Publication
(10) Pub. No.: US 2024/0316180 A1
(43) Pub. Date: Sep. 26, 2024

(54) ATTENUATED MUMPS VACCINE AND USES THEREOF

(71) Applicant: Korea National Institute of Health, Cheongju-si (KR)

(72) Inventors: Tae-young LEE, Cheongju-si (KR); Seo Yeon KIM, Cheongju-si (KR); Hyeran WON, Cheongju-si (KR); You-Jin KIM, Cheongju-si (KR); Dokeun KIM, Cheongju-si (KR)

(21) Appl. No.: 18/516,257

(22) Filed: Nov. 21, 2023

(30) Foreign Application Priority Data
Mar. 21, 2023 (KR) 10-2023-0036649
Nov. 13, 2023 (KR) 10-2023-0156123

(51) Int. Cl. A61K 39/165; A61P 31/14; A61P 37/04; C12N 7/00; G01N 33/569

(52) U.S. Cl. CPC A61K 39/165; A61P 31/14; A61P 37/04; C12N 7/00; G01N 33/56983; A61K 2039/5254; C12N 2760/18721; C12N 2760/18722; C12N 2760/18734; G01N 2333/12

(57) ABSTRACT

The present invention relates to an attenuated mumps virus and a live vaccine comprising the same. The attenuated mumps virus vaccine of the present invention can be effectively used for additional vaccination to control the occurrence of breakthrough infection and the epidemic of mumps due to differences in genotypes of existing vaccine strains and epidemic strains and decreased immunity.

### CROSS-REFERENCE TO RELATED APPLICATIONS

This application claims priority under 35 U.S.C. § 119 to KR 10-2023-0036649, filed on Mar. 21, 2023 and to KR 10-2023-0156123, filed on Nov. 13, 2023, the entirety of each of which are incorporated herein by specific reference for all purposes.

### SEQUENCE LISTING STATEMENT

Incorporated herein by reference in its entirety is a Sequence Listing named “2023FPO-07-010US_sequence_listing”, which is being submitted to the United States Patent and Trademark Office via Patent Center on even date herewith as an XML format 17 KB in size. This file, which was created on Nov. 13, 2023, constitutes both the paper and computer readable form of the Sequence Listing.

### Statement on Deposit of Microorganisms

Under the provisions of the Budapest Treaty on the International Recognition of the Deposit of Microorganisms for the Purpose of Patent Procedure, Applicant has deposited the isolated mumps virus disclosed herein with the Korean Collection for Type Cultures; Korea Research Institute of Bioscience and Biotechnology (KRIBB), 181, Ipsin-gil, Jeongeup-si, Jeolllabuk-do 56212, Republic of Korea, as International Depository Authority, on Feb. 21, 2023 under the accession number: KCTC 15330BP. Submitted herewith is form BP/4 (KCTC Form 17) for the RECEIPT IN THE CSSE OF AN ORIGINAL DEPOSIT issued pursuant to Rule 7.1 of the regulations Under the Budapest Treaty on the International Recognition of the Deposit of Microorganisms for the Purposes of Patent Procedure.

### BACKGROUND OF THE INVENTION

### Field of the Invention

The present invention relates to an attenuated mumps virus and a live vaccine comprising the same.

### Description of the Related Art

Mumps is an acute febrile disease characterized by swelling of the parotid gland caused by infection with mumps virus, also called epidemic parotitis. When infected with the mumps virus, there is typically an incubation period of about 1 to 2 weeks, followed by 1 to 2 days of early symptoms such as fever, headache, muscle pain, loss of appetite, and nausea. Afterwards, the parotid gland swells and feels pain, and the pain becomes most severe about 1 to 3 days after the salivary glands start to swell, and then gradually subside. The pain of parotitis can make chewing and swallowing very painful, and it can also lead to a loss of appetite.

The incidence of mumps has drastically decreased as vaccination against measles, mumps, and rubella (MMR) has become more common. However, mumps outbreaks continue to be reported even in countries where the mumps vaccine has been introduced. The occurrence of this breakthrough infection is attributed to various factors, but the primary reasons are believed to be waning immunity and cross-defense impairment due to genetic differences between epidemic strains (genotypes F, H, I and G) and a vaccine strain (genotype A).

In Korea, the number of mumps cases has decreased significantly since the introduction of the second dose of MMR vaccine in 1997, but more than 15,000 cases have been reported since 2013, and the age of onset is reported to be 3-8 years old and 15-17 years old. In cases of outbreaks within the community, accelerated vaccinations for children and additional vaccinations for adults are recommended, and the MMR vaccine is used during this period. In a previous study, it was confirmed that an effective immune response was induced in the group that received the second dose of the domestically prevalent strain F genotype inactivated mumps vaccine than in the group (animal model) that received the second dose of the current vaccine strain, Jeryl Lynn. Therefore, there is a need to develop a pandemic-ready booster vaccine that responds to various genotypes and induces consistently high neutralizing capacity in case of a community outbreak according to the current vaccination policy.

According to the results of the domestic ‘2014 Measles, Mumps, and Rubella Immunity Survey’, the positive rate of antibodies to mumps virus after MMR vaccination was 74% in 2-3 year olds, 86% in 4-6 year olds, and 89% in 7-9 year olds. However, the antibody positivity rate for individuals aged 13 and older decreased to 62%, which coincided with the age group (13-18 years old) where mumps was more prevalent in 2014. Immunity survey results from overseas also report that there is a decrease in antibody level and a difference in avidity of mumps IgG over time after vaccination. Therefore, the possibility of a decline in immunogenicity after vaccination (waning immunity) has been suggested as a possible explanation for the increased incidence of mumps despite high vaccination rates.

In addition to waning immunity, a decrease in cross-protection ability due to genetic differences in mumps virus strains is predicted to be the cause of breakthrough infection. The mumps virus belongs to Rubulavirus, Paramyxovirinae, Paramyxoviridae, and contains approximately 15.3 kb of negative sense RNA. The structure is arranged from the 3′ end in the following order: nucleoprotein (NP), phospho protein (P), matrix protein (M), fusion protein (F), small hydrophobic Protein (SH), hemagglutinin-neuraminidase protein (HN), and large protein (L). The mumps virus is classified into (A, B, C, D, F, G, H, I, J, K, L, and N) genotypes based on the diversity of the SH and HN genes. The mumps virus is known to be prevalent in European and American regions with the genotype G, while in East Asian countries, the genotypes F, H, and I are predominant. In Korea, the genotypes H and I were prevalent between 2007 and 2014, the genotype F was partially prevalent in 2008, and the genotypes H and I were mainly prevalent since 2015. The current domestic commercial mumps vaccine strain (Jeryl-Lynn, RIT 4385) is the genotype A, which is genetically different. Therefore, it is expected that a combination of these factors, including the genetic differences between vaccine strains and epidemic strains, as well as waning immunity, contributes to breakthrough infections.

In the prior art, Korean Patent Publication No. 10-2021-0083604 discloses the development of a vaccine candidate strain by inactivating a domestic epidemic strain (genotype F) isolated from a patient sample with formaldehyde and confirming its excellent cross-neutralizing ability. In EP4056687, an attenuated vaccine was produced by subculturing a recombinant mumps virus with a deletion of SH gene of the genotype F, and it was confirmed that the attenuated vaccine had excellent immunogenicity. In addition, Yan Liang et al. (Microbes and Infection 12, 2010, 1178-1187) developed an attenuated vaccine by subculturing the genotype F virus in KMB-17, human embryonic lung diploid cells, about 30 passages and confirmed the efficacy of the vaccine. When human diploid cells were used for producing vaccines, the vaccines showed weak immunogenicity, but were safe to use (The Journal of Infectious Diseases, 2019; 219:50-8). For producing attenuated mumps vaccines, the human diploid cells were not suitable since it had the disadvantage of low sensitivity and low production yields using human diploid cells.

Therefore, in order to develop a mumps vaccine candidate, the present inventors completed the present invention by securing a vaccine candidate targeting a genotype of an epidemic strain other than the genotype currently used as a vaccine strain and confirming its immunogenicity. In the present invention, it was confirmed that attenuated F genotype mumps vaccine produced using Vero cells had high immunogenicity as well as low neurotoxicity.

### SUMMARY OF THE INVENTION

It is an object of the present invention to provide a novel mumps virus.

It is another object of the present invention to provide a vaccine composition for preventing or treating mumps comprising the mumps virus.

It is another object of the present invention to provide a diagnostic kit for mumps virus comprising the mumps virus or its antigen.

It is another object of the present invention to provide a method for producing the mumps virus.

It is another object of the present invention to provide a method for inducing immunity containing a step of administering the vaccine composition.

To achieve the above objects, the present invention provides a mumps virus (Accession Number: KCTC 15330BP).

The present invention also provides a vaccine composition for preventing or treating mumps comprising the mumps virus.

The present invention also provides a diagnostic kit for mumps virus comprising the mumps virus or its antigen.

The present invention also provides a method for producing an attenuated mumps virus containing a step of subculturing the mumps virus in Vero cells.

In addition, the present invention provides a method for inducing immunity containing a step of administering the vaccine composition for preventing or treating mumps comprising the mumps virus.

### Advantageous Effect

The present invention relates to an attenuated mumps virus and a live vaccine comprising the same. The attenuated mumps virus vaccine of the present invention can be effectively used for additional vaccination to control the occurrence of breakthrough infection and the epidemic of mumps due to differences in genotypes of existing vaccine strains and epidemic strains and decreased immunity.

### BRIEF DESCRIPTION OF THE DRAWINGS

FIG. 1 is a diagram showing the measurement of mumps virus in MRC5, WI38, and Vero cells using focus formation assay.

FIG. 2 is a diagram showing the method for producing an attenuated genotype F mumps vaccine.

FIG. 3 is a diagram showing the cell lesions according to subculture of attenuated vaccine candidate strains.

FIG. 4 is a graph showing the growth curve of each virus.

FIG. 5 is a graph comparing and analyzing the plaque size of each virus.

FIG. 6 is a set of photographs comparing the plaque shape of each virus.

FIG. 7 is a graph showing the quantification of brain inflammation induced by each virus.

FIG. 8 is a set of photographs confirming the inflammatory response in the brain induced by each virus through H&E staining.

FIG. 9 is a diagram showing the method for immunizing mice with the vaccine candidate strain.

FIG. 10A is a graph analyzing the binding antibody titer in mice immunized with the vaccine candidate strain.

FIG. 10B is a graph showing the quantification of the binding antibody titer in the serum of mice immunized with the vaccine candidate strain.

FIG. 11 is a graph analyzing the neutralizing antibody titer in mice immunized with the vaccine candidate strain alone.

FIG. 12 is a graph analyzing the ability to induce IFN-7 in mice immunized with the vaccine candidate strain alone.

FIG. 13 is a graph analyzing the neutralizing antibody titer in mice booster-immunized with the vaccine candidate strain.

FIG. 14 is a graph analyzing the ability to induce IFN-7 in mice booster-immunized with the vaccine candidate strain.

FIG. 15 is a diagram showing the method of challenging the mumps virus to mice immunized with the Jeryl-Lynn strain and then boosted with the vaccine candidate strain.

FIG. 16 is a graph analyzing the protective efficacy against genotype F of mumps virus infection in mice immunized with the vaccine candidate strain.

FIG. 17 is a graph analyzing the protective efficacy against genotype G of mumps virus infection in mice immunized with the vaccine candidate strain.

### DESCRIPTION OF THE PREFERRED EMBODIMENTS

Hereinafter, the present invention is described in detail.

The present invention provides a mumps virus (Accession Number: KCTC 15330BP).

The mumps virus is an attenuated virus obtained by infecting Vero cells with the genotype F mumps vaccine strain and then subculturing the cells 30 passages. The virus can be represented by SEQ. ID. NO: 1.

The virus may have thirteen nucleotide sequence changes and nine amino acid changes compared to the initial virus strain (F0). In one embodiment of the invention, T was mutated to A at 14, G was mutated to A at 503, C was mutated to T at 1738, G was mutated to A at 4777, A was mutated to G at 5350, T was mutated to C at 6437, A was mutated to T at 6586, A was mutated to G at 7073 and 9349, C was mutated to A at 8105, A was mutated to G at 9349, C was mutated to A at 10889, A was mutated to G at 12654 and C was mutated to A at 14274. In addition, Ala was mutated to Thr at amino acid position 120 in the nuclear protein (NP), Asp was mutated to Asn at amino acid position 78 and Met was mutated to Val at amino acid position 269 in the Fusion protein (F), Leu was mutated to Pro at amino acid position 57 in the small hydrophobic protein (SH), Thr was mutated to Ala at amino acid position 154 and His was mutated to Asn at amino acid position 498 in the hemagglutinin-neuraminidase protein (HN), and His was mutated to Asn at amino acid position 818, Lys was mutated to Arg at amino acid position 1406, and Pro was mutated to Gln at amino acid position 1946 in the large protein (L).

The virus may have nine nucleotide sequence changes and five amino acid changes compared to the virus strain subcultured 10 passages (F10). In one embodiment of the invention, A was mutated to G at nucleotide position 11, T was mutated to A at nucleotide position 14, G was mutated to A at nucleotide position 503, C was mutated to T at 1738, T was mutated to C at 6437, A was mutated to G at 7073 and 9349, and C was mutated to A at 10889 and 14274. In addition, Ala was mutated to Thr at amino acid position 120 in the nuclear protein (NP), Leu was mutated to Pro at amino acid position 57 in the small hydrophobic protein (SH), Thr was mutated to Ala at amino acid position 154 in the hemagglutinin-neuraminidase protein (HN), His was mutated to Asn at amino acid position 818 at amino acid position 818 in the large protein (L), and Pro was mutated to Gln at amino acid position 1946.

The present invention also provides a vaccine composition for preventing or treating mumps comprising the mumps virus (Accession Number: KCTC 15330BP) as an active ingredient and one or more pharmaceutically acceptable carriers.

The pharmaceutically acceptable carrier can be selected or be prepared by mixing more than one ingredients selected from the group consisting of saline, Ringer's solution, buffered saline, dextrose solution, maltodextrose solution, glycerol, and ethanol. Other general additives such as anti-oxidative agents, buffer solution, bacteriostatic agents, etc., can be added. In order to prepare injectable solutions such as aqueous solution, suspension and emulsion, diluents, dispersing agents, surfactants, binders, and lubricants can be additionally added. The vaccine composition of the present invention can further be prepared in suitable forms for each disease or according to ingredients by following a method represented in Remington's Pharmaceutical Science (the newest edition), Mack Publishing Company, Easton PA.

In the vaccine composition, the mumps virus may be included at a concentration of 1×10¹ pfu to 1×10¹⁰ pfu, and preferably may be included at a concentration of 1×10³ pfu to 1×10⁷ pfu. More preferably, the virus may be included in the vaccine composition at a concentration of 1×10⁵ pfu, but not always limited thereto.

The dosage of the vaccine composition may vary depending on the individual's weight, age, gender, health condition, diet, administration time, administration method, and severity of disease, and can be administered in one or several divided doses.

The vaccine composition can be administered by any one or more routes selected from the group consisting of oral, transdermal, intramuscular, intraperitoneal, intradermal, subcutaneous, and nasal routes, and preferably by intramuscular route, but not always limited thereto.

The vaccine composition of the present invention can be a standalone vaccine or a booster vaccine. In one embodiment of the present invention, it was confirmed that an effective immune response was induced in mice immunized with the vaccine composition alone, and an effective immune response was also induced in mice immunized with a booster. Therefore, the vaccine composition of the present invention can be used alone or as a booster vaccine.

The present invention also provides a diagnostic kit for mumps virus comprising the mumps virus or its antigen.

The mumps virus or its antigen of the present invention can be used not only to eliminate mumps virus in cells to be infected or infected through an antigen-antibody complex reaction, but also to specifically detect the mumps virus.

The diagnostic kit can include a virus sample containing the mumps virus of the present invention and a reagent for detecting the antigen-antibody complex. The reagent for detecting the antigen-antibody complex can contain reagents for radioimmunoassay, ELISA (enzyme-linked immunosorbent assay), or immunofluorescence assay, and tools, reagents, and the like commonly used in the art for immunological assay.

The present invention also provides a method for producing an attenuated mumps virus containing a step of subculturing the mumps virus in Vero cells.

In addition, the present invention provides a method for inducing immunity containing a step of administering a vaccine composition for preventing or treating mumps comprising the mumps virus (Accession Number: KCTC 15330BP).

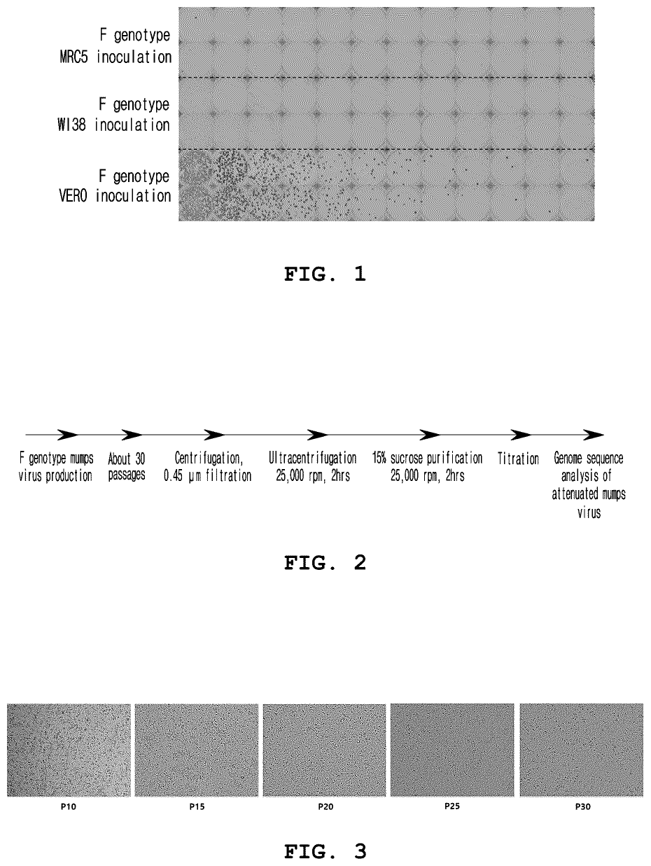

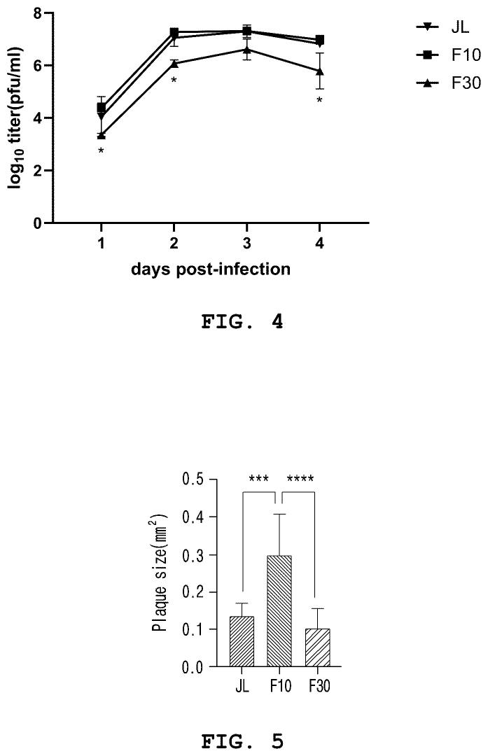

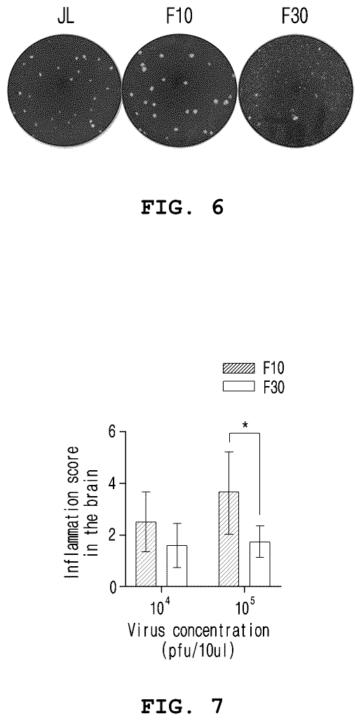

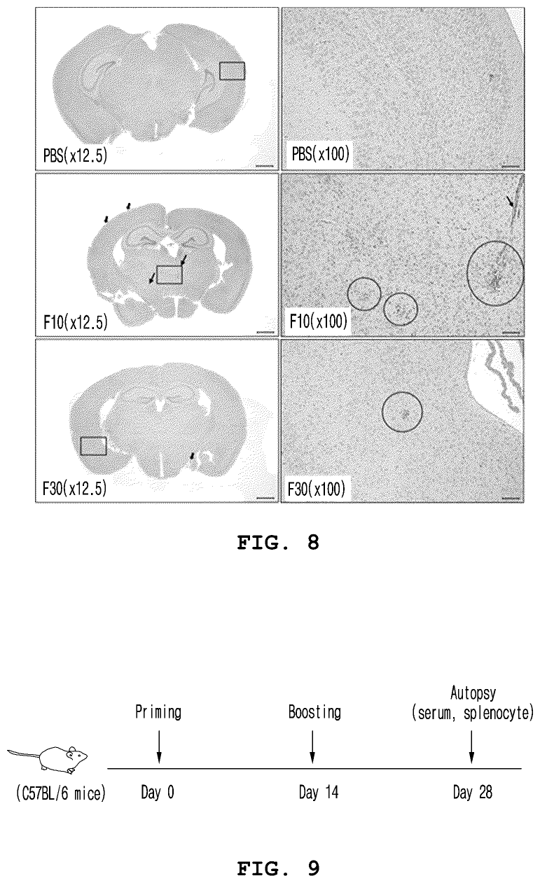

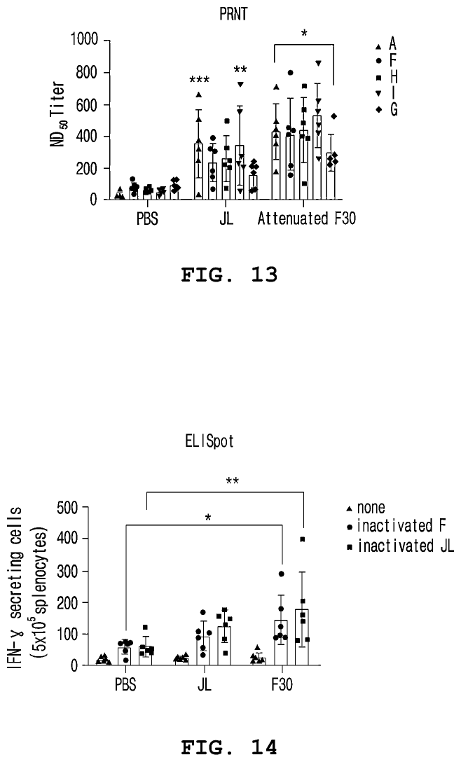

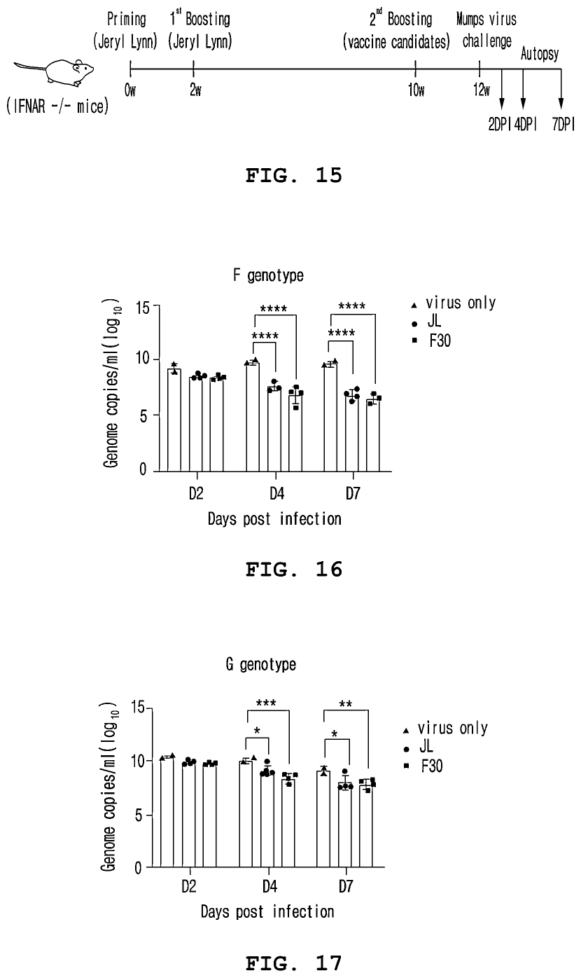

In specific examples and experimental examples of the present invention, Vero cells were infected with the genotype F mumps virus and then subcultured 30 passages to produce the attenuated mumps virus (see FIG. 2). As a result of focus formation assay, it was confirmed that the attenuated vaccine candidate (F30) was highly infectious compared to the virus produced in human diploid cells, MRC5 and WI38 cells (see FIG. 1), and the cellular lesions were confirmed in morphology (see FIG. 3). It was also confirmed that the vaccine candidate strain produced in Vero cells had higher immunogenicity than that of the vaccine produced in MRC5 and WI38 cells, so the vaccine candidate strain produced in Vero cells was further analyzed. First, the nucleotide sequence and amino acid sequence of the attenuated vaccine candidate strain (F30) were analyzed through full-length analysis. As a result, it was confirmed that the attenuated candidate strain (F30) with 30 passages had nine nucleotide sequence changes and five amino acid changes compared to the initial virus strain (F10) with 10 passages (see Table 1). The attenuated virus strain (F30) had about 10 times lower titer than that of the existing vaccine strain JL (Jeryl-Lynn) and the initial virus strain (F10) prepared by subculturing the genotype F10 passages, but the growth rate was confirmed to be similar (see FIG. 4). In addition, the plaque size of the attenuated candidate strain (F30) was significantly smaller than that of the initial virus strain (F10) and similar to the existing vaccine strain (JL) (see FIGS. 5 and 6). Meanwhile, the attenuated candidate strain (F30), a live virus vaccine, was tested for neuropathogenicity. As a result, it was confirmed that the inflammatory response caused by the attenuated candidate strain (F30) was reduced compared to the inflammatory response caused by the initial virus strain (F10), indicating that neurotoxicity was reduced (see FIGS. 7 and 8). In addition, the immunogenicity of the attenuated vaccine candidate strain alone was evaluated using a mouse model. As a result, it was confirmed that the attenuated candidate strain (F30) showed high binding antibody titer and high neutralizing antibody titer (see FIGS. 9, 10A, 10B, and 11). It was also confirmed that the attenuated candidate strain (F30) could induce a humoral immune response, and that it had excellent IFN-7 induction ability (see FIG. 12), indicating that it could induce a cellular immune response. In addition, the existing vaccine strain, Jeryl Lynn (genotype A), was subjected to secondary immunization, followed by a tertiary boosting with the attenuated vaccine candidate strain to evaluate the booster immunogenicity of the attenuated vaccine candidate strain. As a result, it was confirmed that the attenuated candidate strain (F30) showed high neutralizing antibody titers against all genotypes of viruses (A, F, H, I, and G) (see FIG. 13) and also induced a high cellular immune response (see FIG. 14). Furthermore, the protective efficacy of the candidate vaccine strain against mumps virus infection by immunizing immunocompromised mice (IFNAR KO mice) with the existing vaccine strain, Jeryl Lynn (genotype A) and then boosting with the vaccine candidate strain. As a result, the vaccine candidate strain (F30) showed a higher protective efficacy against genotypes F and G of the mumps virus (see FIGS. 16 and 17).

Therefore, the attenuated vaccine candidate of the present invention can be effectively used as a vaccine for preventing mumps.

Hereinafter, the present invention will be described in detail by the following examples and experimental examples.

However, the following examples and experimental examples are only for illustrating the present invention, and the contents of the present invention are not limited thereto.

### Example 1: Acquisition and Production of Mumps Virus Vaccine Candidate

A genotype F attenuated mumps vaccine candidate was produced.

Producing vaccine candidates in human diploid cells has the advantage of verifying safety, so MRC5 and WI38 cells were infected with the genotype F mumps virus, but as a result of focus formation assay, it was confirmed that infection was hardly detected in both cell types (FIG. 1).

Due to the low susceptibility of MRC5 and WI38 cells to mumps virus infection, Vero cells were infected with the genotype F mumps virus and then subcultured 30 passages to attenuate them (FIG. 2).

Specifically, when a 75T flask was 90-100% full of Vero cells, 1 ml of the genotype F mumps virus stored at −70° C. was added thereto and incubated for 1 hour in a 37° C., 5% CO₂ incubator, while shaking well to prevent the cells from drying out. After 1 hour, the virus was removed, and the medium was replaced with 10 ml of DMEM containing 2% FBS and 1% penicillin-streptomycin, followed by culture for 3 days in a 37° C., 5% CO₂ incubator. Once cellular lesions were identified, the flask was frozen at −70° C. Before the next subculture, the flask was thawed and the virus culture solution was harvested using a cell scraper, followed by centrifugation (2,000 rpm, 15 minutes, 4° C.). Only the supernatant was taken and aliquoted 1 ml into each cryo vial tube and stored at −70° C. This process was considered to be one passage number and was repeated.

The cell shape of the attenuated genotype F mumps vaccine candidate strain was observed, and the infection was confirmed by focus formation assay.

Specifically, Vero cells were seeded in a 96-well plate at the density of 2×10⁴ cells/well. After culturing the cells for 24 hours, the virus was serially diluted two-fold using MEM containing 2% FBS and 1% penicillin-streptomycin. The diluted virus was inoculated into Vero cells and cultured in a 37° C., 5% CO₂ incubator for 1 hour. After 1 hour of culture, the virus culture medium was removed, and an overlay medium (MEM containing 2% FBS, 1% penicillin-streptomycin and 1.5% carboxymethylcellulose) was added thereto, followed by culture for 2 days. For cell fixation, 100% methanol was added thereto and left at room temperature for 30 minutes. After washing with PBS, a blocking buffer (PBS containing 1% BSA, 0.1% FBS, and 0.1% Tween-20) was added thereto and left at room temperature for 30 minutes. After removing the blocking buffer, the primary antibody (1:1000, anti-mumps antibody, Ab9880) was added thereto and cultured at room temperature for 1 hour. After washing with a washing buffer (PBS containing 0.1% Tween-20, PBST), the secondary antibody (1:2000, Goat anti-Mouse IgG, HRP conjugate) was added thereto and reacted at room temperature for 1 hour. After washing with PBST, the chromogenic substrate (TrueBlue peroxidase substrate) was dispensed and left until spots appeared, then the substrate was removed and dried. At this time, blue spots were formed in cells infected with mumps virus due to the chromogenic substrate. The formed spots were counted using CTL automated equipment.

As a result, as shown in FIG. 3, morphological cell lesions were confirmed in Vero cells upon subculture. In addition, as a result of focus formation assay, it was confirmed that infection occurred in Vero cells, unlike MRC5 and WI38 cells, as shown in FIG. 1.

The above results suggest that the vaccine candidate strain produced in Vero cells is more immunogenic than those produced in MRC5 and WI38 cells.

### Experimental Example 1: Characteristic Analysis of Attenuated Vaccine Candidates

### <1-1> Comparative Analysis of Full-Length Sequences

The nucleotide sequence and amino acid sequence of the attenuated candidate strain (F30) subcultured 30 passages in Vero cells produced in Example 1 were analyzed through full-length analysis.

As a result, as shown in Table 1, it was confirmed that the attenuated candidate strain (F30) subcultured 30 passages had thirteen nucleotide sequence changes and nine amino acid changes compared to the initial virus (F0) strain which had not subcultured and had nine nucleotide sequence changes and five amino acid changes compared to the virus strain (F10) subcultured 10 passages. The changed sites were nuclear protein (NP), small hydrophobic protein (SH), hemagglutinin-neuraminidase protein (HN), and large protein (L).

TABLE 1

| Gene | Nucleotide | Nucleotide | | | Amino acid | Amino acid | | |
|---|---|---|---|---|---|---|---|---|
| ID | position | F0 | F10 | F30 | position | F0 | F10 | F30 |
| — | 11 | — | A | G | — | — | — | — |
| — | 14 | T | T | A | — | — | — | — |
| NP | 503 | G | G | A | 120 | Ala | Ala | Thr |
| NP | 1,738 | C | C | T | 531 | Thr | Thr | Thr |
| F | 4,777 | G | A | A | 78 | Asp | Asn | Asn |
| F | 5,350 | A | G | G | 269 | Met | Val | Val |
| SH | 6,437 | T | T | C | 57 | Leu | Leu | Pro |
| — | 6,586 | A | T | T | — | — | — | — |
| HN | 7,073 | A | A | G | 154 | Thr | Thr | Ala |
| HN | 8,105 | C | A | A | 498 | His | Asn | Asn |
| L | 9,349 | A | A | G | 304 | Gin | Gln | Gln |
| L | 10,889 | C | C | A | 818 | His | His | Asn |
| L | 12,654 | A | G | G | 1406 | Lys | Arg | Arg |
| L | 14,274 | C | C | A | 1946 | Pro | Pro | Gln |

### <1-2> Phenotypic Characterization of Attenuated Vaccine Candidates

Growth curves were measured to compare the growth of the attenuated virus strains.

Specifically, Vero cells were infected with the existing vaccine strain JL (Jeryl-Lynn), the initial virus strain (F10) prepared by subculturing the genotype F 10 passages, and the attenuated candidate strain (F30) subcultured 30 passages at 0.01 MOI, and the plaque morphology was observed daily.

As a result, as shown in FIG. 4, it was confirmed that the titer was increased significantly between 1 day post infection (dpi) and 2 dpi for all virus strains. When compared by time, it was confirmed that the titer of the attenuated candidate strain (F30) was about 10 times lower than that of JL or F10.

Through the above results, it was confirmed that the growth rate and virus titer of the attenuated candidate strain (F30) were lower than those of the initial virus.

### 5<1-3> Comparison of Morphology of Attenuated Vaccine Candidates

To compare the morphology of the attenuated viral strains, the sizes of the top 10 plaques were compared.

Specifically, the plaque morphology of the virus strains was compared through plaque forming assay. Vero cells infected and cultured with the existing vaccine strain (JL), the initial virus strain (F10), and the attenuated candidate strain (F30) at the same MOI were stained with crystal violet to confirm the plaque formation. The size of the top 10 plaques was measured using the ImageJ program.

As a result, as shown in FIGS. 5 and 6, it was confirmed that the size of the plaque of the attenuated candidate strain (F30) was significantly reduced compared to that of the initial virus strain (F10). It was also confirmed that the size of the plaque of the attenuated candidate strain (F30) was similar to or smaller than that of the existing vaccine strain (JL).

The above results suggest that the plaque sizes of the attenuated candidate strain (F30) of the present invention and the existing vaccine strain (JL) are similar.

### <1-4> Evaluation of Neurovirulence

A neurotoxicity evaluation was performed because there are reports that mumps virus has a neuropathogenic risk and the attenuated vaccine is a live virus vaccine.

Specifically, young mouse brains were inoculated with the attenuated candidate strain by the intracranial (IC) route at different concentrations and encephalopathy was confirmed after 25 days by brain tissue staining. Tissue processing was performed by transversely cutting three areas from the front of the brain fixed in 10% formalin solution. Paraffin-embedded blocks were prepared, sectioned at 3 μm thickness, and stained with hematoxylin and eosin (H&E). The stained tissue was observed under an optical microscope, and scores were given according to the degree of staining. The main lesions observed in the brain tissue were multifocal microgliosis, perivascular cuffing, and leptomeningitis. Scores were given and evaluated according to the frequency and severity of each lesion, and the combined inflammatory index values were compared.

As a result, as shown in FIGS. 7 and 8, it was confirmed that the inflammatory response of the attenuated candidate strain (F30) was reduced compared to the inflammatory response of the initial virus strain (F10).

The above results suggest that the attenuated candidate strain (F30) of the present invention exhibits lower encephalopathy compared to the initial viral strain (F10).

### Experimental Example 2: Evaluation of Independent Immunogenicity of Attenuated Vaccine Candidate Strains

The immunogenicity of the attenuated vaccine candidate strain alone was evaluated using a mouse model.

Specifically, C57BL/6 mice (4 weeks old, female) were intramuscularly inoculated (I.M.) with the attenuated vaccine candidate strain twice at a concentration of 1×10⁵ pfu two weeks apart, and then serum and splenocytes were obtained to measure humoral and cellular immunity (FIG. 9).

### <2-1> Confirmation of Binding Antibody Titer

The binding antibody titer was measured to evaluate whether the virus induces a humoral immune response.

Specifically, a mixture of live mumps virus genotypes (A, F, H, I, and G) was seeded in a 96 well plates at a final concentration of 5×10⁴ ffu/well and left overnight at 4° C. Afterwards, an equal amount of 10% skim milk solution was added to the live virus mixture, and blocking was performed for 1 hour at 37° C. with a final 5% skim milk solution. After washing with washing buffer (PBS+0.05% Tween-20, PBST), serum serially diluted 2-fold in 3% skim milk was dispensed onto the coated plate and reacted at 37° C. for 2 hours. After washing with wash buffer, the secondary antibody (goat anti-mouse IgG, HRP conjugate) diluted 1:5,000 was added thereto and reacted at 37° C. for 1 hour. After washing again, TMB substrate solution was added thereto and reacted at room temperature for 10 minutes, and then stop solution (2 M H₂SO₄) was added to terminate the reaction. Absorbance was measured at a wavelength of 450 nm using a microplate reader, and the binding antibody titers were calculated based on the wavelength and compared between experimental groups.

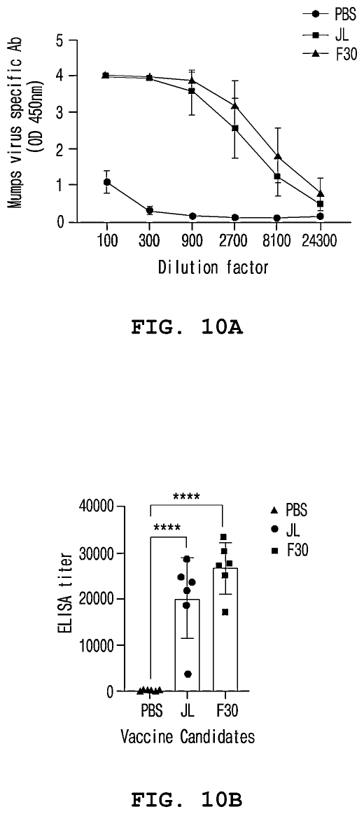

As a result, as shown in FIGS. 10A and 10B, the high binding antibody titer of the attenuated candidate strain (F30) was confirmed.

### <2-2> Confirmation of Neutralizing Antibody Titer

The neutralizing antibody titer was measured to evaluate whether the virus induces a humoral immune response.

Specifically, the serum of a singly immunized mouse inactivated at 56° C. was serially diluted two-fold in a 96-well plate with MEM containing 2% FBS and 1% penicillin-streptomycin. A neutralization step was performed by inoculating the mumps virus of various genotypes (A, F, H, I, and G) diluted to 400 pfu/ml at 1:1 with the diluted serum and culturing for 1 hour in a 37° C., 5% CO₂ incubator. One day before the experiment, the neutralized serum-virus mixture was inoculated into a 24-well plate seeded with Vero cells at 1×10⁵ cells/well and cultured for 1 hour in a 37° C., 5% CO₂ incubator. After removing the serum-virus mixture, an overlay medium (MEM containing 2% FBS, 1% penicillin-streptomycin, and 1.5% carboxymethylcellulose) was added thereto and cultured for 6 days. After removing the overlay medium, crystal violet dye was added thereto and left for 2 hours. After removing the dye, the plate was dried thoroughly and the plaques were counted to calculate the neutralizing antibody titer.

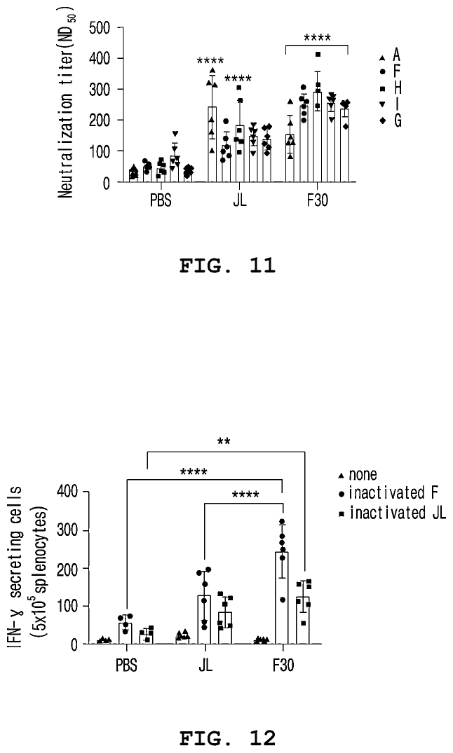

As a result, as shown in FIG. 11, the neutralizing antibody titer of the attenuated candidate strain (F30) to all genotypes of viruses (A, F, H, I, and G) was significantly higher compared to PBS, and the neutralizing antibody titer to all genotypes except the genotype A was higher than that of the existing vaccine strain, JL strain.

### <2-3> Confirmation of IFN-Gamma Inducing Ability

To evaluate whether a cellular immune response was induced, ELISpot was performed to measure T cells secreting virus-specific IFN-7 in splenocytes of singly immunized mice.

Specifically, the spleens of mice immunized with the vaccine were isolated in RPMI containing 5% FBS and 1% penicillin-streptomycin, and the spleens were pulverized into single cells using gentleMACS equipment. Splenocytes were filtered using a 70 μm strainer and centrifuged (2,000 rpm, 5 minutes, 4° C.). The supernatant was removed, 5 ml of ACK Lysing buffer was added to the remaining pellet, vortexed, and left at 37° C. for 5 minutes. Another 10 ml of RPMI containing 5% FBS and 1% P/S was added thereto, and centrifuged under the same conditions. The supernatant was discarded, 1 to 3 ml of RPMI containing 10% FBS and 1% P/S was added thereto to resuspend the pellet well, and splenocytes were counted using a LUNA cell counter. Splenocytes were seeded in a 96-well plate in the Mouse IFN-7 ELISpot plus kit (MABTECH) at a concentration of 5×10⁵ cells/well. As a stimulant, inactivated mumps virus of genotype A and F was diluted to a final concentration of 1 μg/well and dispensed into each well of the plate. The cell-stimulant mixture was incubated for 12-48 hours in a 37° C., 5% CO₂ incubator. The cell-stimulant mixture was removed, washed with PBS, and the primary antibody diluted in PBS-0.5% FBS solution was added thereto, followed by culture at room temperature for 2 hours. After washing as above, the ALP-labeled secondary antibody diluted in PBS-0.5% FBS solution was added thereto, followed by culture at room temperature for 1 hour. After washing again, a filter-sterilized substrate solution (BCIP/NBT-plus) was added thereto and reacted for 10-30 minutes until spots appeared. To terminate the reaction, the plate was washed with distilled water and dried well. The generated spots were counted using CTL automated equipment.

As a result, as shown in FIG. 12, it was confirmed that the attenuated candidate strain (F30) induced a high cellular immune response.

### Experimental Example 3: Evaluation of Booster Immunogenicity of Attenuated Vaccine Candidate Strains

The immunogenicity of the attenuated vaccine candidate strain was evaluated using a mouse model.

Specifically, a group of C57BL/6 mice (4 weeks, female) secondarily immunized with the existing vaccine strain, Jeryl-Lynn (genotype A), were inoculated intramuscularly (I.M.) with the attenuated vaccine candidate strain twice at a concentration of 1×10⁵ pfu two weeks apart, followed by a third boost of the attenuated vaccine candidate strain at a concentration of 1×10⁵ pfu eight weeks later. Then, serum and splenocytes were obtained to measure humoral and cellular immunity.

### <3-1> Confirmation of Neutralizing Antibody Titer

Neutralizing antibody titers were measured to evaluate whether a humoral immune response was induced.

Specifically, the serum of a booster immunized mouse inactivated at 56° C. was serially diluted two-fold in a 96-well plate with MEM containing 2% FBS and 1% penicillin-streptomycin. A neutralization step was performed by inoculating the mumps virus of various genotypes (A, F, H, I, and G) diluted to 400 PFU/ml at 1:1 with the diluted serum and culturing for 1 hour in a 37° C., 5% CO₂ incubator. One day before the experiment, the neutralized serum-virus mixture was inoculated into a 24-well plate seeded with Vero cells at 1×10⁵ cells/well and cultured for 1 hour in a 37° C., 5% CO₂ incubator. After removing the serum-virus mixture, an overlay medium (MEM containing 2% FBS, 1% penicillin-streptomycin, and 1.5% carboxymethylcellulose) was added thereto and cultured for 6 days. After removing the overlay medium, crystal violet dye was added thereto and left for 2 hours. After removing the dye, the plate was dried thoroughly and the plaques were counted to calculate the neutralizing antibody titer.

As a result, as shown in FIG. 13, the neutralizing antibody titer of the attenuated candidate strain (F30) to all genotypes of viruses (A, F, H, I, and G) was significantly higher compared to PBS, and the neutralizing antibody titer to all genotypes was higher than that of the existing vaccine strain, JL strain.

### <3-2> Confirmation of IFN-Gamma Inducing Ability

To evaluate whether a cellular immune response was induced, ELISpot was performed to measure T cells secreting virus-specific IFN-γ in the splenocytes of booster immunized mice.

Specifically, the spleens of mice immunized with the vaccine were isolated in RPMI containing 5% FBS and 1% penicillin-streptomycin, and the spleens were pulverized into single cells using gentleMACS equipment. Splenocytes were filtered using a 70 μm strainer and centrifuged (2,000 rpm, 5 minutes, 4° C.). The supernatant was removed, 5 ml of ACK Lysing buffer was added to the remaining pellet, vortexed, and left at 37° C. for 5 minutes. Another 10 ml of RPMI containing 5% FBS and 1% P/S was added thereto, and centrifuged under the same conditions. The supernatant was discarded, 1 to 3 ml of RPMI containing 10% FBS and 1% P/S was added thereto to resuspend the pellet well, and splenocytes were counted using a LUNA cell counter. Splenocytes were seeded in a 96-well plate in the Mouse IFN-γ ELISpot plus kit (MABTECH) at a concentration of 5×10⁵ cells/well. As a stimulant, inactivated mumps virus of genotype A and F was diluted to a final concentration of 1 μg/well and dispensed into each well of the plate. The cell-stimulant mixture was incubated for 12-48 hours in a 37° C., 5% CO₂ incubator. The cell-stimulant mixture was removed, washed with PBS, and the primary antibody diluted in PBS-0.5% FBS solution was added thereto, followed by culture at room temperature for 2 hours. After washing as above, the ALP-labeled secondary antibody diluted in PBS-0.5% FBS solution was added thereto, followed by culture at room temperature for 1 hour. After washing again, a filter-sterilized substrate solution (BCIP/NBT-plus) was added thereto, and reacted for 10-30 minutes until spots appeared. To terminate the reaction, the plate was washed with distilled water and dried well. The generated spots were counted using CTL automated equipment.

As a result, as shown in FIG. 14, it was confirmed that the attenuated candidate strain (F30) induced a significantly higher cellular immune response and had a higher immune inducing capacity than the existing vaccine strain, JL.

### Experimental Example 4: Evaluation of Protective Immunity of Attenuated Vaccine Candidate Strains Against Mumps Virus

The protective immunity of the attenuated vaccine candidate strain was evaluated using a mouse model.

Specifically, a group of IFNAR knock-out mice (4 weeks, female) were secondarily immunized with the existing vaccine strain, Jeryl-Lynn (genotype A) and boosted with the attenuated vaccine candidate strain at a concentration of 1×10⁵ Pfu eight weeks later. Two weeks later, the mice were inoculated with Mumps live virus at a concentration of 3×10⁶ Pfu. Then, RNA was extracted from the supernatant obtained by PBS solution with homogenized lung tissue of mice collected on 2, 4, and 7 days from the inoculation. To detect mumps virus-specific genes, the number of viral genome copies was analyzed by PowerChek™ Mumps Virus Real-Time PCR Kit Ver.1.0(cat #: R3110C) with the extracted RNA (FIG. 15).

As a result, it was confirmed that the attenuated candidate strain (F30) had a higher protective immunity against infection of genotype G as well as genotype F of mumps virus (FIGS. 16 and 17).

1. An isolated mumps virus, comprising amino acid variants of
Ala to Thr at amino acid position 120 in the nuclear protein (NP),
Asp to Asn at amino acid position 78 and Met to Val at amino acid position 269 in the fusion protein (F),
Leu to Pro at amino acid position 57 in the small hydrophobic protein (SH),
Thr to Ala at amino acid position 154 and His to Asn at amino acid position 498 in the hematogglutin-neuraminidase protein (HN), and
His to Asn at amino acid position 818, Lys to Arg at amino acid position 1406, and Pro to Gln at amino acid position 1946 in the large protein (L).

2. The isolated mumps virus of claim 1, comprising a polynucleotide consisting of the nucleotide sequence of SEQ ID NO: 1.

3. The isolated mumps virus of claim 1, wherein the virus is as deposited with accession number: KCTC 15330BP.

4. The isolated mumps virus of claim 1, wherein the virus is genotype F.

5. The isolated mumps virus of claim 1, wherein the virus is an attenuated virus.

6. The isolated mumps virus of claim 5, wherein the virus is Vero cell subcultured attenuated virus.

7. The isolated mumps virus of claim 6, wherein the virus is subcultured in Vero cells more than 30 passages.

8. A vaccine composition, comprising:
the isolated mumps virus of claim 1 as an active ingredient; and
one or more pharmaceutically acceptable carriers.

9. The vaccine composition of claim 8, wherein the vaccine composition is suitable as a stand-alone vaccine or a booster vaccine.

10. The vaccine composition of claim 8, wherein the content of the isolated mumps virus is in the range of 1×10¹ pfu to 1×10¹⁰ pfu.

11. The vaccine composition of claim 10, wherein the content of the isolated mumps virus is in the range of 1×10³ pfu to 1×10⁷ pfu.

12. The vaccine composition of claim 8, wherein the vaccine composition can be used for MMR (measles, mumps, and rubella) combination vaccine.

13. A diagnostic kit for mumps virus comprising the mumps virus of claim 1 or its antigen and reagents for detecting the antigen-antibody complex.

14. The diagnostic kit of claim 13, wherein the reagents for detecting the antigen-antibody are the reagents used for radioimmunoassay, ELISA (enzyme-linked immunosorbent assay), or immunofluorescence assay.

15. A method for producing an attenuated mumps virus, the method comprising subculturing the isolated mumps virus of claim 1 in Vero cells.

16. A method for inducing immunity against mumps virus infection, the method comprising administering the vaccine composition of claim 8 to a subject.

17. An isolated mumps virus comprising a polynucleotide consisting of the nucleotide sequence of SEQ ID NO: 1.

18. An isolated mumps virus as deposited under accession number: KCTC 15330BP.


SEQUENCE LISTING (1 sequence)

SEQ ID NO: 1 - DNA, 15385 bp, unidentified
  source 1..15385: mol_type = genomic DNA
accaaggggagaaagaagatgggatattggtagaacaaatagtgtaagaaacagtaagcccggaagtggtgttttgcgatttcgaggccgggctcgatcctcaccttccattgtcgatagaggatattttgacactatctggaaaatgtcgtctgtgctcaaagcatttgagcgattcacgatagaacaggaactacaagacaggggtgaggagggttcaattccgccggagactttaaagtcagcagtcaaagtcttcgtcattaacacacccaatcccaccacacgctaccagatgctaaacttttgcctaagaataatctgcagtcaaaatgctagggcgtctcacagggtaggtgcattgataacattattctcacttccctcagcaggcatgcaaaatcatattagattagcagatagatcacccgaagctcagatagaacgctgtgagattgatggttttgagcccggcacatataggctgatcccgaatgcacgcaccaatcttactgccaatgaaattgccgcctatgctttgcttgcagatgacctccctccaaccataaataatggaactccttatgtacatgcagatgttgaaggtcagccatgtgatgagattgagcaattcctggatcgatgctacagtgtactaatccaggcttgggtgatggtctgtaaatgtatgacagcgtacgaccaacctgctgggtctgctgatcggcgatttgctaagtaccagcagcaaggtcgcctggaggctagatacatgctgcagccggaggcccaaaggttgattcaaactgccatcaggaaaagtcttgttgttaggcagtaccttaccttcgaactccagctggcaagacggcagggattgctgtcaaacagatactatgcaatggtaggtgacattggaaagtacattgagaattcaggccttactgccttctttctcaccctcaaatacgcactaggtactaaatggagtcctttgtcattggccgcattcaccggtgaactcactaagctccgatccttgatgatgttgtatcgggatctcggtgaacaagccagataccttgctttgttggaggctccccaaataatggactttgcacccgggggctacccactgatattcagttatgccatgggagtcggtacagtcctagatgtccaaatgcgaaattacacttacgcacgacctttcctaaatggttattacttccagattggggttgagactgcaagacggcaacaaggcactgttgacaacagagtagcagatgatctaggcctgactcctgagcaaagaactgaggttactcagcttgttgacaggcttgcaaggggcagaggtgcagggataccaggtggacctgtgaatccatttgttcctccagttcaacaacaacaacctgctgctgtatatgaggacattcctgcattggaggaatcagatgacgatggcgatgaagatggaggtgcaggattccaaaatggagcacaagcaccagctgtaagacagggaggtcaaaatgactttagagtacagccattacaggatccaattcaagcacagcttttcatgccactatatcctcaagtcagcaacatcccaaaccatcagaatcatcaggtcaatcgcatcggggggatggaacaccaagatttattacgatacaacgagaatggtgattctcaacaagacgcgaggggcgagcaaggaaatactttcccaaacaatcccaatcaaaacacacagtcgcaagtgggcgactgggatgagtaaattactgacatgatcaaactaccccctaatgcaattaccccaggacaatctagccacagctaactgcccaaatccactacattctatccatatttagtctttaagaaaaaattaggcccggaaagaattagttctacgaacatcgacaaagttatcttgatcgtgtttctttccgggcaagccatggatcaatttataaaacaagatgagactggtgatttaattgagacaggaatgaacgttgcaaatcatttcctatccgcccccattcagggaaccaactcgttgggcaaagctacaatcatccctggtgttgcaccagtactcattggcaatccagatcaaaaaaacattcaataccccaccgcatcacatcagggatccaaatcaaagggcagaagctcagggaccaggcccatcatagtctcatcttccgaagggggcactggagggactcagattcctgagccccttttcgcacaaacaggacaaggtggcattgtcaccaccgtttatcaggatccaacgatccaaccaacaggttcataccgaagtgtggaattggctaagataggaaaagagagaatgattaatcgatttgttgaaaaacccagaacctcaacgccggtgacagaatttaagaggggggccgggagcggctgctcaaggccagataatccaagaggagggcatagacgggaatggagcctcagctgggtccaaggagaggtccgggtctttgagtggtgcaaccccatatgctcacctatcactgccgcagcaagattccactcctgcaaatgtgggaattgccccgcaaagtgcgatcagtgcgaacgagattatggacctccttagagggatggatgctcgtctgcaacatcttgaacaaaaggtggacaaggtgcttgcacagggcagcatggtgacccaaataaagaatgaattatcaacagtaaagacaacactagctacaattgagggaatgatggcgaccgtaaagatcatggatcctggaaacccgacaggggtcccagtcgatgagcttagaagaagtttcagtgatcatgtaacaattattagtggaccaggagatgtgtcattcagctccggcgaagagcccacactatatttggatgaactagcaaggcccgtccccaagcctcgtcctgcaaagcagccaaaaccccaaccagtaaaggatttagcaggacggaaagtgatgataaccaaaatgatcactgactgtgtggccaaccctcaaatgaagcaggtgtttgagcaacgactggcaaaggccagcaccgaggatgccctgaatgacatcaagcgagacatcatacgaagcgccatatgaatcaaccagaaacacaagacctaaggggaaatccacaaattaagagccacaatgattccctgattaataaaaaataagcacgaacacaagtccaatccaaccatagcagcaatggccggatcacagatcaaaattcctcttccaaagccccccgattcagactcccaaagactaaatgcattccctgtaattatggcccaagaaggcaaaggacgactcctcagacagatcagacttaggaaaatattatcaggggatccgtctgatcagcaaattacatttgtgaatacatatggattcatccgtgccactccagaaacatccgagttcatctctgaatcatcacaacaaaaggtgactcctgtagtgacggcgtgcatgctgtccttcggtgctggaccagtactagaggacccacaacatatgctaaaagctcttgaccagacagacatcagggttcggaagacagcgagtgataaagagcagatcttgttcgagatcaaccgtatccccaatctattcaggcatcatcaaatatctgcggaccatctgattcaggccagctccgataaatatgtcaagtcaccagcaaagttgattgcaggagtaaattacatctactgtgtcacatttttatctgtgacagtgtgttctgcctcactcaagtttcgagttgcgcgcccattgcttgctgcacgatctagattagtaagagcagttcagatggaaattttgcttagggtaacttgcaagaaagattcccaaatggcaaagagcatgttaaatgaccctgatggcgaagggtgcattgcatccgtgtggttccacctgtgtaatctgtgcaaaggcaggaataaacttagaagttacgatgaaaattattttgcatctaaatgccgtaagatgaacctgacagtcagcataggagacatgtggggaccaaccattctagtccatgcaggcggtcacattccgacaactgcaaaaccttttttcaactcaagaggctgggtctgccaccccatccaccaatcatcaccatcgttggcgaagaccctatggtcatctgggtgcgaaatcaaggctgccagtgctatcctccagggctcagattatgcatcactcgcaaaaactgatgacataatatactcaaagataaaagtcgataaagatgctgccaactacaagggtgtatcctggagtccattcaggaagtctgcctcaatgagcaacctatgaaaatttcctctattcccacggatgcctccaggaggatcaacaatcagtccgatttgaccggtggtaacttgattgaaattatagaaaaaataagcctagaaggatatcttacttcttgactttccaactttgaaaagagaataaatcagtaatcatgaaggcttttttggttatttgcttgggctttgcagtcttttcatattctatctgtgtgaatatcaacatcttgcagcaagttggatatatcaagcaacaagtcaggcaactaagttattactcacaaagttcaagctcctacatagtggtcaagcttttaccgaatatccaacccactgacaacagctgtgaatttaagagtgtaactcaatataataagaccttgggtaatttgctccttccaattgcagaaaacataaacaatattgcatcgccctcacctgggtcaagacgtcataaaaggtttgctggcattgccatcggcattgctgcgctcggtgttgcgaccgcagcacaagtaactgccgctgtctcattagttcaagcacagacaaatgcacgtgcgatagcggcaatgaaaaattcaatacaggcaactaatcgagcagtcttcgaagtgaaagaaggaactcaacagttagctatagcggtacaagcaatacaagaccacatcaatactattatgaacactcaattgagcaatatgtcttgtcagatccttgataaccagcttgcgacttccctaggattatacctaacagaattaacaacagtgtttcagccccaattaattaatccggcactgtcaccgattagtatacaagccttgaggtctttgcttggaagtatgacacctgcagtggttcaagcaacattatctacttcaatttctgctgctgaaatactaagtgccggtctaatggagggtcagattgtgtctgttctgctagatgagatgcaggtgatagttaagataaatattccaaccattgtcacacaatcaaatgcattggtgattgacttctactcaatttcgagctttattaataatcaggaaaccataattcaattaccagacaggatcttggagatcgggaatgaacagtggagctatccagctaaaaattgtaagttgacaagacacaacatattttgccaatacaatgaggcagagaggctgagcctagaatcaaaactatgccttgcaggcaatataagtgcctgtgtgttctcacccatagcagggagttatatgaggcgatttgtagcgctggatggaacaattgttgcaaactgtcgaagtctaacgtgtctatgcaagagtccatcttatcctatataccaacctgaccatcatgcagtcacaaccattgatctaaccgcatgtcaaacattgtccctagacggattggatttcagcattgtctctctaagcaacatcacttacgctgagaaccttaccatttcattgtctcagacaatcaatactcaacccattgacatatcaactgaactaagtaaagttaatgcatccctccaaaatgccgttaagtacataaaagagagcaaccaacaactccaatctgtgagtgtaaattctaaaatcggagctataattatagcagccttagttttgagcatcctgtcaattatcatttcgctattgttttgctgctgggcctacattgcaactaaagaaatcaggagaatcaacttcaaaacaaatcatatcaacacaatatcaagtagtgtcgatgatctcatcaggtactaatcctaacattgtgattcatcctgcattgagaaaagatttagaaaaaaaccaaattaagaatgaatctcctggggtcgtaacgtctcgtgaccctgccgttgcactatgccggcaatccaccctcccttatacctaatatttctattgctaattcttcttcatctgatcataactttgtatgtctggattctgttaaccattacttacaagactgcggtgcaacatgcagcactgtaccatagatccctctttcgttggagtttcgatcactcaccctagaaagatctccaacccggacaagtcccaatccataatgggagagcaagctgcattcaaatgatgctgttcaatcatgagacataaagaaaaaagcaggccagaacaaactcaggatcacaacataacacagaaccccagctgccatcacaactgtgctccagcagcctaaaagatggagccttcgaaactcttcacaatatcggacaatgccacctttgcacctgggcctgttatcaatgcagctgacaagaagacattccgaacctgcttccgaatattggtactgtctgtacaagctgttacccttatattagtcattgtcactttaggtgagcttgtgaggatgatcaatgatcaaggcttgagtaatcagttgtcttcaattacaaacaagataagagagtcagctactatgattgcatctgctgtgggagtaatgaatcaagttattcacggagtgacgatatccttacccctacaaattgagggaaaccaaaatcaattgttatccacacttgccacaatctgtacaagcaaaaaacaagtctcaaactgctctacgaatatccccttagttaatgaccttaggtttataaatgggatcaataaattcatcattgaagattattcagctcatgatttctccatcggccatccactcaacatgcctagctttatcccaactgcaacctcacccaatggttgcacaaggattccatccttctctctaggtaagacacactggtgctacacacataatgtaattaatgccaactgcaaggatcatacttcgtctaaccaatatgtttccatggggattctcgttcagaccgcgtcagggtatcctatgttcaaaaccttaaaaatccaatatctcagtgatggcctgaatcggaaaagctgctcaattgcaacagtccctgatggatgcgcaatgtattgttacgtctcaactcaacttgaaaccgacgactatgcagggtccagcccacctacccagaaacttaccctgttgttctataatgacaccgtcacagaaaggacaatatctccatctggtcttgaagggaattgggctactttggtgccaggagtggggagtggaatatatttcgagaataaattgatctttcctgcatatgggggtgttttgcccaatagtacacttggagttaagtcagcaagagaatttttccggcctgttaatccatataatccatgttcaggaccacaacaagatttagatcagcgtgctttgagatcatacttcccaagttacttctctaatcgaagagtgcagagtgcattccttgtctgtgcttggaatcagattctagttacaaattgcgagctagttgtcccctcaaacaatcaaacactgatgggtgcagaaggaagagttttattgatcaataatcgattattatattatcagagaagtaccagctggtggccgtatgaactcctctatgagatatcattcacctttacaaactctggtcaatcgtctgtgaatatgtcctggatacctatatactcattcactcgtcctggttcaggtaactgcagtggtgaaaatgtgtgcccgactgcttgtgtgtcaggggtttatcttgacccttggccattaactccatatagccaccaatcaggcattaacagaaatttctatttcacaggtgcactattaaattcaagcacaaccagagtaaatcctaccctttatgtttctgcccttaataatcttaaagtactagccccatatggtactcaaggactgtttgcctcgtacaccacaaccacctgctttcaagataccggtgatgctagtgtgtattgtgtttatattatggaactagcatcgaatatcgttggagaattccaaattctacctgtgctaaccagactgaccatcacttgagtcatagtgaatgcagcggtaggccctatgggagtgtcccaagttttatcgattattaagaaaaaacaggccagaatggcgggcctaaatgagatactcttacctgaagtacatttgaactcacccatcgttagatataagcttttctactatatattgcatggccagttaccaaatgatttggagccagatgacttgggcccactagcaaatcagaattggaaggcaattcgatctgaagaatcccaggttcatgcacgtttaaaacagatcagagtagaactcattgcaaggattcctagtctccggtggacccgctctcaaagagagattgccatactcatttggccaagaatacttccaatcctgcaagcatatgatcttcgacaaagtatgcaattgcccacagtatgggagaaattgactcaatccacagttaatcttataagtgatggcctagaacgggttgtattacatatcagcaatcaactgacaggcaagcctaacttgtttaccagatctcgagcaggacaagacgcaaaggattcctcaattccatccactagagagctatctcaaatatggtttaacaatgagtggagtggatctgtaaagacctggcttatgattaaatatagaatgaggcagctaatcacaaaccaaaagacaggtgagttaacagatttagtaaccattgtggatactaggtccactctatgcattattaccccagaattagttgctttatactccagtgagcacaaagcattaacgtacctcacctttgaaatggtattaatggtcactgatatgttagagggacgactgaatgtttcttctttatgcactgctagtcattatctgtcccctctaaagaagagaatcgaaattctcctaacattagttgatgaccttgctctactcatgggggataaagtatacggtgttgtctcttcacttgagagttttgtttatgcccaattacagtatggtgatcctgttgtagacattaagggcacattctatggatttatatgtaatgagattctcgacctactgacggaagacaacatctttactgatgaggaggcacacaaggttcttctggacttgacatcacagtttgacaatctatcccctgatttaactgctgagctcctctgcattatgagactttggggccatcccacactaactgccagccaagcagcatccaaggtccgagagtccatgtgcgctcctaaggtattagatttccaaacaataatgaagacgctggctttctttcacgcaatcctgattaacggttataggaggagccataatggaatctggcctcctactactcttcatggcaatgcccccaaaagcctcattgagatgcgccatgataattcagagcttaagtatgaatatgtcctcaagaattggaaaagtatatctatgttaaggatacacaaatgctttgatgcatcacccgatgaagatctcagcatattcatgaaggataaggcaataagctgtccaaagcaagactggatgggagtatttaggagaagcctgataaaacagcgatatcgtgatgcgaatcggcctctaccacaaccattcaaccgacggctactgttgaattttctagaggatgacagattcgatcctattaaagagcttgagtatgtcaccagtggagaatatcttagggaccctgaattttgtgcatcttattctctcaaggagaaggagataaaggctacaggtcgtatatttgcgaaaatgacaaagagaatgagatcgtgccaagtaattgcagagtcattgttggccaatcatgcaggaaaattaatgagagagaatggagttgtcttagaccagttaaaattgacaaaatctttgttaacgatgaaccaaattggtattatatcagagcacagccgaagatccaccgctgacaacatgactttggcacactccggttccaataagcacagaattaataatagtcaattcaagaagaataaagacaataaacatgagatgcctgatgatgggtttgagatagcagcctgcttcctaacaactgacctcacaaaatactgcttaaattggaggtaccaagttatcatcccctttgcgcgtacattgaactcaatgtatggtataccccatctgttcgaatggatacatttaaggctaatgcgaagcactctctatgtcggtgatcccttcaatcctccatcagatcctacccaacttgaccttgatacagctctcaacgatgatatatttatagtttctcctcgtggaggaatcgaggggttatgtcaaaaattatggactatgatttccatctcaacaattatattatccgcaactgaggcaaacactagagttatgagcatggttcagggtgacaaccaagcaattgcaatcaccactagagtagtacgttcgctcagtcattccgagaagaaggagcaagcttataaagcgagtaaattattctttgaaaggcttagagctaacaacaatggaattggacaccacttaaaagaacaagaaacaatccttagttctgatttcttcatatacagtaagagggtgttttacaaaggtcgaattttgactcaggcgttaaagaatgtgagcaagatgtgcttaacagctgatatactgggggactgctcacaagcatcatgctccaatttagccactactgtaatgcgcctgactgagaacggggtcgagaaagatttgtgttattttctaaatgcattcatgacaatcagacaattatgttatgatctagtatttccccaaactaaatctcttagtcaggacatcactaatgcttatctcaatcatccaatacttatctcaagattgtgtctattaccatctcaattggggggcctaaattttctctcgtgtagtcgcctgttcaatagaaacataggagacccattagtgtctgcaattgctgatgtgaaacgattaattaaagctggctgtttagacatctgggtcttatataacatccttggaagaagacctgggaaaggtaagtggagcactctggcagctgatccctatacgttaaacatagattatttagttccttcaacaacttttttaaagaagcatgcacaatatacattgatggagcggagtgttaatccaatgctccgtggagtattcagcgaaaatgcagctgaggaagaggaggtactcgcacagtatctattagatcgtgaggtagtcatgccaagagttgcacatgtaatacttgcccagtctagttgcggtagaagaaagcagattcaaggttacttggattccactaggactattatcaggtattcattggaggtgagaccattgtcagcaaagaagctgaatacagttatagaatataacttattgtatctgtcctataatttggagattattgaaaaacccaatatagtccagccttttttgaatgcaatcaatgttgatacttgtagcatagatatagctaggtcccttagaaaactatcctgggctactttacttaatggacgtcccatcgagggattagaaacacctgatcccattgaattggtacatgggtgtctaataattgggtcagatgaatgtgaacattgcagtagtggtgatgacaagtttacctggtttttcctacccaaggggataaggctagatagtgacccggcatccaacccacccatcagagtaccttatattggatctaaaacagatgagcggagggttgcgtcaatggcttacatcaaaggagcatctgtatcacttaaatcagcactcaggttagcgggggtatacatttgggctttcggagatacagaggaatcatggcaagatgcctatgagttagcgtccactcgtgttaatctcacgctagagcaattgcaatctctcactcctttgccaacatctgccaacctagtccacagattggatgatggtactactcaattaaaatttaccccggcaagctcttatgcattctctagctttgtgcatatatctaatgactgtcaagttcttgagatcgatgatcaggtaacagattctaacctgatttaccagcaagttatgattactggccttgcgttaattgagacatggaacaatcctccaattaacttctccgtttatgaaactacactacacctgcacacaggctcatcttgctgtatcagacctgttgagtcttgtgtagtaaatcctcctttgcttcctgtccccttcattaatgttcctcaaatgaataggtttgtatatgaccctgaaccactcagtttgctagagatggaaaaaattgaggatattgcttatcaaaccagaattggcggtttagatcaaatcccacttctggaaaaaatacccttactagctcacctcaccgccaagcagatggtaaatagcatcactgggcttgatgaagcaacatctatagtgaatgacgctgtagttcaagcagactatactagcaattggattagtgaatgctgctacacttatattgattctgtgtttgtttactctggctgggcattattattggaactttcgtaccaaatgtactacttaagaattcaaggcatccaaggaattatagactatgtgtatatgaccttgaggaggataccaggaatggctataacaggcatctcatccacaatcagtcaccctcgtatactcagaagatgcatcaatttggatgtcatagccccaatcaattctccacacatagcttcactagattacacaaaattgagcatagatgcagtaatgtggggaactaagcaggttttgaccaacatttcgcaaggtatcgattatgagatagtcgttccttctgaaagccagctcacactcagtgatagagttctaaatctagtcgctcgaaaattatcactacttgcaatcatctgggccaattataactatcctccaaaggttaaaggcatgtcacctgaggacaaatgtcaggctttaactacacacctactccagactgtcgagtatgttgagcacattcagattgaaaagacaaacatcaggaggatgattattgaaccaaaattaactgcctaccctagtaatttgttttatctatctcgaaagttgcttaatgctattagagactctgaagaaggacaattcctgattgcatcttattataacagttttggatatttggaaccaatactaatggaatctaaaatattcaatctaagttcatctgaatcagcatctcttacagagttcgatttcatcctcaacttggaattgtctgaaaccagccttgagaaatactctctcccaagtttgcttatgacggctgagaatatggataacccatttcctcaacccccccttcatcatgttctcagaccattaggtttatcatccacctcatggtataaaacaatcagtgttttgaattatattagccatatgaagatatctgacggtgcccatctatacttggcagagggaagtggagcctctatgtcacttatagagactttcttgcccggtgaaacaatatggtacaacagcctattcaatagtggtgagaatcctccccaacgcaatttcgcccctttgcccacccagttcattgaaagtgtcccttacagattgattcaagcaggtatagcagcaggaagtggtgtagtgcaaagtttctatccactctggaacggtaacagcgacatcactgacttaagcacgaaaactagtgtcgaatacattattcacaaggtgggagcagatacatgtgcattggttcatgtggatttggagggtgtacccggctcaatgaatagcatgctggaaagagcccaagtacatgcgctactgatcacggtaactgtattaaagcaaggtggcttactaatcttgaaagcttcatgggaaccttttaatcgattttcctttttactcacaatactctggcaattcttttcaacaataagaatcctgcgatcttcatactctgacccgaataatcacgaggtatacataatagctacattagcagttgatcccaccacatcctcctttacaactgctctgaatagggcgcgcaccctgaatgaacagggcttttcactcatcccacctgaattagtgagtgagtactggaggaggcgcgttgaacaagggcagattatacaggatcgtatagataaagtcatatcagaatgtgtcagagaccaatacctgtcagacaacaacattatccttcaggcaggagggactccaagcacaagaaaatggttggatctacctgactatccttcgttcaatgaattacaatcggagatggccagactcataacaattcatctcaaagagataatagaaatcctaaaaggccaatcatcggatcatgacaccttattatttacttcatacaatgtaggtcccctcgggaaaataaatacaatactcagattgattgttgagagaattcttatgtacactgtaaggaactggtgcatcttgcccacccaaactcgtctcaccttacgacagtctatcgagcttggagagtttagactaagagacgtgataacacccatggagatccttaagctatcccccaacaggaaatatctgaagtctgcattaaaccaatcaacattcaatcatctaatgggtgaaacatccgacatattgttaaatcgagcctatcagaagagaatttggaaagccattggatgtgtaatctattgctttggtttgcttaccccggatgttgaagattccgagcgcattgatattgataatgatatacctgattatgatatccacggggacataatttaaatcggttacagactcctctggtatgatacatcaccaaaaggtgccacaccagcatcccaattcttctagaccgcacacgacctcgaacaatcataaccacatcagtattaaatccataatatcattttaagaaaaaattgatttaactttctccccttggat